(19) United States
(12) Patent Application Publication
(10) Pub. No.: US 2025/0170186 A1
(43) Pub. Date: May 29, 2025

(54) MEDICAMENT FOR MENSTRUAL AND ENDOMETRIOSIS PROBLEMS

(71) Applicant: Vernon Patrick MANCER, Germiston (ZA)

(72) Inventor: Vernon Patrick MANCER, Germiston (ZA)

(21) Appl. No.: 18/956,445

(22) Filed: Nov. 22, 2024

(30) Foreign Application Priority Data
Nov. 23, 2023 (ZA) 2023/06499

(51) Int. Cl. A61K 35/618; A61K 31/375; A61K 33/00; A61K 33/04; A61K 33/06; A61K 33/26; A61K 33/30; A61K 33/34; A61K 36/54; A61P 15/00; A61P 29/00

(52) U.S. Cl. CPC A61K 35/618; A61K 31/375; A61K 33/00; A61K 33/04; A61K 33/06; A61K 33/26; A61K 33/30; A61K 33/34; A61K 36/54; A61P 15/00; A61P 29/00

(57) ABSTRACT

The use of Seawater Pearl Powder in the manufacture of a medicament for treating a woman suffering from menstrual pains and or irregular menstrual symptoms and or endometriosis.

This application is based on South African Provisional Patent Application No. 2023/06499 filed on 27 Oct. 2023 by the same inventor.

This invention relates to a medicament for the treatment of menstrual problems. The medicament may be used for treating menstrual problems and in particular for pain medication and especially for patients suffering from endometriosis.

As is known endometriosis is particularly painful. Endometriosis affects roughly 10% (190 million) of reproductive age women and girls globally. It is a chronic disease associated with severe, life impacting pain during periods, sexual intercourse, bowel movements and/or urination, chronic pelvic pain, abdominal bloating and nausea, fatigue and sometimes depression, anxiety and infertility. There is currently no known cure for endometriosis and treatment is usually aimed at controlling symptoms.

Access to early diagnosis and effective treatment of endometriosis is important but is limited in many settings including in low- and middle-income countries.

Endometriosis is a disease in which tissue similar to the lining of the uterus grows outside the uterus and can cause severe pain in the pelvis and makes it harder to get pregnant.

Endometriosis can start at a woman's first menstrual period and last until menopause. According to the invention there is provided the use of Seawater Pearl Powder in the manufacture of a medicament for treating a women having menstrual problems and in particular endometrioses.

The medication preferably further comprises other medicinal powders preferably cinnamon powder and vitamin C powder. The seawater pearl powder is preferably ground to superfine powder. The cinnamon powder is likewise ground to fine powder.

According to one aspect of the current invention there is provided a medicament comprising Seawater Pearl Powder for treating a woman suffering from menstrual pains and or irregular menstrual symptoms.

According to a further aspect of the current invention there is provided a medicament comprising Seawater Pearl Powder for treating a woman suffering from endometrioses.

It has been found to be advantageous to include Vitamin C in the medicament.

It has further been found to be advantageous to include cinnamon in the medicament.

An embodiment of the invention will now be described by way of example.

A young lady aged 32 was a diagnosed as suffering from endometriosis. After 30 months when conventional treatment was ineffective, she commenced treatment with medication of the invention.

The said medication comprises 70 mg Seawater Pearl Powder, 10 mg cinnamon powder, 10 mg vitamin C powder. The Seawater Pearl Powder was ground in to superfine powder. The other powders are similarly ground to a fine powder. The Seawater Pearl Powder was oxygenated for plus minus one hour. The Seawater Pearl Powder is oxygenated by the use of an Ozone generator. The Seawater Pearl Powder absorbed the oxygen until saturated. The oxygenated Seawater Pearl Powder was then mixed the other powders and thereafter was filled into 90 mg capsules.

Vitamin C powder assists the body to absorb calcium (which is the main component of Seawater Pearl Powder). Cinnamon powder reduces swelling and discomfort.

The Seawater Pearl Powder comprises:—selenium 1.2 mg/g; germanium 0.1 mg/g; zinc 9.3 mg/g; iron 2.0 mg/g; copper 0.3 mg/g; magnesium 11.3 mg/g; calcium 89-92 mg/g; trace elements 1-3 mg/g and also protein 4,5-8.3 mg/g comprising aspartic acid; serine; Arginine; Threonine; Alanine; Methionine; isoleucine; phenylalanine; Lysine and glutamic acid. The Seawater Pearl Powder is ground to a superfine powder.

The patient took one 90 mg capsule per day for three months. After three months she suffered no more pain and terminated taking the capsules. After five months she fell pregnant.

The medication has proven is a safe, natural and effective treatment of Endometriosis.

A further patient had severe menstrual pain during her menstrual cycle. One capsule was taken every day of the menstrual cycle. Virtually from the first day of taking the medication, her pain subdued and she was virtually pain free.

The invention is not limited to the precise details hereinbefore described. For example, the Seawater Pearl Powder of the invention may comprise 88% to 90% natural calcium and the other 10% to 12% trace elements.

1. The use of Seawater Pearl Powder in the manufacture of a medicament for treating a woman suffering from menstrual pains and or irregular menstrual symptoms.

2. The use of Seawater Pearl Powder in the manufacture of the medicament for treating a woman suffering from endometrioses.

3. A medicament as claimed in claim 1 further comprising at least one other medicinal powder.

4. A medicament as claimed in claim 3 wherein one of the said other powders comprise cinnamon powder.

5. A medicament as claimed in claim 3 wherein one of the said other powders comprise vitamin C powder.

6. A medicament as claimed in claim 3 wherein the said other powders comprise vitamin C powder and cinnamon powder.

7. A medicament as claimed in claim 5 comprising between 60% w/w and 90% w/w Seawater Pearl Powder and between 10% w/w and 30% w/w Vitamin C Powder.

8. A medicament as claimed in claim 6 comprising between 60% w/w and 90% w/w Seawater Pearl Powder and a combination of cinnamon and Vitamin C Powder of between 10% w/w and 30% w/w.

9. A medicament as claimed in claim 1 wherein the Seawater Pearl Powder comprises:—selenium 1.2 mg/g; germanium 0.1 mg/g; zinc 9.3 mg/g; iron 2.0 mg/g; copper 0.3 mg/g; magnesium 11.3 mg/g; calcium 89-92 mg/g; trace elements 1-3 mg/g and also protein 4,5-8.3 mg/g

10. A medicament as claimed in claim 9 wherein the protein comprises aspartic acid; serine; Arginine; Threonine; Alanine; Methionine; isoleucine; phenylalanine; Lysine and glutamic acid.

11. A method of treatment of the woman suffering from menstrual pains and or irregular menstrual symptoms comprising ingesting a 90 mg capsule containing the medicament as claimed in claim 1.

12. A medicament as claimed in claim 2 further comprising at least one other medicinal powder.

13. A medicament as claimed in claim 12 wherein one of the said other powders comprise cinnamon powder.

14. A medicament as claimed in claim 12 wherein one of the said other powders comprise vitamin C powder.

15. A medicament as claimed in claim 12 wherein the said other powders comprise vitamin C powder and cinnamon powder.

16. A medicament as claimed in claim 14 comprising between 60% w/w and 90% w/w Seawater Pearl Powder and between 10% w/w and 30% w/w Vitamin C Powder.

17. A medicament as claimed in claim 15 comprising between 60% w/w and 90% w/w Seawater Pearl Powder and a combination of cinnamon and Vitamin C Powder of between 10% w/w and 30% w/w.

18. A method of treatment of the woman suffering from endometriosis comprising ingesting a 90 mg capsule containing the medicament as claimed in claim 2.

19. A medicament as claimed in claim 2 wherein the Seawater Pearl Powder comprises:—selenium 1.2 mg/g; germanium 0.1 mg/g; zinc 9.3 mg/g; iron 2.0 mg/g; copper 0.3 mg/g; magnesium 11.3 mg/g; calcium 89-92 mg/g; trace elements 1-3 mg/g and also protein 4,5-8.3 mg/g

20. A medicament as claimed in claim 19 wherein the protein comprises aspartic acid; serine; Arginine; Threonine; Alanine; Methionine; isoleucine; phenylalanine; Lysine and glutamic acid.
